(19) United States
(12) Patent Application Publication
(10) Pub. No.: US 2020/0254056 A1
(43) Pub. Date: Aug. 13, 2020

(54) MEDICATION FOR TREATING CELIAC DISEASE AND METHOD FOR PRODUCING SAME

(71) Applicant: FEDERAL STATE AUTONOMOUS EDUCATIONAL INSTITUTION OF HIGHER EDUCATION I.M.SECHENOV FIRST MOSCOW STATE, Moscow (RU)

(72) Inventors: Andrey Aleksandrovich ZAMYATNIN, Moscow (RU); Anna Aleksandrovna ZAPYSHNAYA, Noginsk (RU); Liudmila Anatolievna PAVLOVA, Moscow (RU); Liudmila Vladimirovna SAVVATEEVA, Moscow (RU); Vadim Vladimirovich TARASOV, Moscow (RU)

(21) Appl. No.: 16/753,788

(22) PCT Filed: Oct. 5, 2018
(86) PCT No.: PCT/RU2018/050123

§ 371 Date: Apr. 5, 2020

(30) Foreign Application Priority Data
Oct. 5, 2017 (RU) 2017135534

(51) Int. Cl. A61K 38/16; A61K 47/26; A61K 47/32; A61K 47/12

(52) U.S. Cl. CPC A61K 38/168; A61K 47/12; A61K 47/32; A61K 47/26

(57) ABSTRACT

The invention relates to the pharmaceutical industry, and more particularly to a method for producing a drug based on triticain-α for the treatment of celiac disease. The invention provides improved technological properties of triticain-α, which allows creating the most suitable dosage form for its oral administration (hard hypromellose capsules).

### FIELD OF INVENTION

The invention relates to the pharmaceutical industry and can be used to obtain a drug to treat celiac disease.

### STATE OF THE ART

Celiac disease is autoimmune inflammation of the small intestine mucosa with its atrophy in people with genetically determined gluten sensitivity [Recommendations on the creation of regional and all-Russian registries of patients with celiac disease. Experimental & clinical gastroenterology, 2009; 10:148-153 (In Russ.)]

Celiac disease is a common autoimmune disease that occurs in approx. 1% of the world's population. A lifelong strict gluten-free diet is the only effective way of treating such patients that is recognized in the world [Satya Kurada, Abhijeet Yadav, Daniel A. Leffler (2016): Current and Novel Therapeutic Strategies in Celiac Disease//Expert Review of Clinical Pharmacology, http://dx.doi.org/10.1080/17512433.2016.1200463]

Supportive treatment methods have been also proposed, they do not eliminate the cause of the disease, but improve the patient's condition.

Among these methods is a drug containing a vitamin complex, DNA, RNA, and plant extracts [U.S. Pat. No. 8,128,971B1, Mar. 6, 2012]. The preferred dose to treat celiac disease is the administration of 6 capsules of the drug to one patient orally every day during approx. 30 days. It is assumed that using this composition stimulates the growth of intestinal villi, capable of absorbing nutrients more efficiently.

U.S. Pat. No. 8,128,971B1, Mar. 6, 2012, proposes the use of digestive enzymes in a composition comprising amylases, lipases, proteases, and any combination thereof.

U.S. Pat. No. 6,348,495B1, Feb. 19, 2002, proposes the use of alkanoyl derivatives of L-carnitine (L-carnitine acetyl, propionyl, butyryl, isobutyryl, valeryl and isovaleryl).

U.S. Pat. No. 8,119,104B2, May 19, 2011, describes a method for inhibiting the symptoms of celiac disease, which comprises administering monoclonal or polyclonal, monomeric, dimeric or polymeric preparative IgA, which accumulates on the surface of the mucous membrane and is able to prevent retrotranscytosis of gliadin peptides associated with celiac disease and thereby disrupt the disease process. Excipients used in the pharmaceutical industry are mixed with the monomeric, dimeric and polymeric forms of IgA to form a capsule or tablet, followed by coating it with enteric polymers or suppositories.

At the same time, a number of patents provide information on the possibility of using various protein compounds to create drugs to directly treat celiac disease. They include dosage forms for both injection and oral administration.

Robert Paul Anderson et al. (EP2367561A1, Sep. 28, 2011) revealed three dominant peptides that together could be used as an agent in immunotherapy or as a vaccine to modulate the T-cell response to three or more kinds of gluten peptides and, therefore, provide tolerance to gluten, thus allowing celiac disease treatment. This drug is a solution for injection.

U.S. Pat. No. 7,605,150B2, Oct. 20, 2009, shows the feasibility of administering tTGase, which is a trans-glutaminase. The compound reduces the toxic effects of gluten oligopeptides, thereby attenuating or eliminating the effects of gluten. The drug is also a solution for injection.

In continuation of the work on searching for drugs against celiac disease, U.S. Pat. No. 7,943,312B2, May 17, 2011, presents the process of isolation of an enzyme belonging to the EU classification group 3.4.21.26, EC 3.4.14.5, or EC 3.4.15.1 and intended for the treatment of celiac disease.

EP2736525A1, Apr. 4, 2014, discloses oral formulations for the treatment of celiac disease, which contain a mixture of proenzymes or the enzymes ALV001 and ALV002 themselves or a mixture thereof—ALV003. ALV001 is a modified version of the proenzyme form of barley-derived EP-B2, a glutamine-specific endoprotease from germinating barley seeds, ALV002 is a recombinant version of prolyl endopeptidase from the bacterium Sphingomonas capsulata (SC-PEP).

Each of these biologically active substances or their mixture in combination with mannitol, TRIS, sucrose, EDTA, sodium chloride, citric acid, sodium metabisulfite, cysteine and monothioglycerol can be enclosed in a hypromellose capsule, sized 1, 0, or 00, a foil bag, or formed into a tablet. The daily dose of the active composition is from 100 mg to 3 g. During use, it is necessary to dissolve the drug in water or juice and take it with food.

There is “Method for the production of proteins of the family of cysteine proteases of wheat (Triticum aestivum) and protein preparation of triticain-α obtained by this method” [RU No. 2,603,504, Nov. 27, 2016], however, it provides only a method for producing the protein itself, but describes no method for producing a drug suitable for use. The recombinant protein triticain-α is obtained in the form of a lyophilisate from white to grayish-white, with high hygroscopicity. It is poorly removed from the vial, and given that its dose does not exceed 20 mg, this entails inaccurate dosage and large losses. Moreover, it has unpleasant organoleptic characteristics.

But the recombinant protein triticain-α is resistant to the action of gastric juice, which allows it to be used as part of an oral dosage form—a hard hypromellose capsule.

### DISCLOSURE OF INVENTION

The problem solved by the invention is a method of preparation and a composition of the drug from the recombinant protein triticain-α, used for oral administration.

The technical result consists in improving the technological properties of triticain-α, which allows creating a drug and the most suitable dosage form for its oral use.

The posed problem is solved by a method of obtaining a medicinal product to treat celiac disease, which consists in the addition of excipients mannitol and polyvinylpyrrolidone MW 12,600 (PVP) to the Triticain-α protein with the weight ratio of 1:0.5-2:0.5-2, respectively, the composition is carefully mixed, frozen at −70° C. for at least 2 h, lyophilized for 24 h at an evaporator temperature of −50° C. in vacuum of 0.03-0.04 mbar; MCC 102 and Licatab-C, taken in the weight ratio of 0.2-1.1:0.2-1.1, respectively, are put into the mixer, stirred for 25 min until a homogeneous mass appears, to which the lyophilisate is added at the ratio of 1:0.15-0.5 (weight parts) respectively, magnesium stearate is introduced into the resulting mixture at the ratio of 1:0.007-0.015, respectively, mixed in the mixer for 5-15 min with the chopper operating at 1,000-5,000 rpm, then the chopper rotation speed is reduced down to 500 RPM and the stirring is continued for 10-30 min.

To solve this problem, a drug for the treatment of celiac disease based on triticain-α is proposed, characterized in that it additionally contains mannitol, PVP, microcrystalline cellulose 102, Licatab C, magnesium stearate in the following component ratios (weight parts):

| | |
|---|---|
| Triticain-α | 9-20 |
| Mannitol | 5-10 |
| Polyvinylpyrrolidone MW 12,600 | 5-10 |
| Licatab-C | 26-30 |
| Magnesium stearate | 1-5 |
| MCC 102 | to 100 |

### BRIEF DESCRIPTION OF THE DRAWINGS

FIG. 1 shows the qualitative characteristics of the mixture of the recombinant protein triticain-α obtained by stirring the mixture at a temperature of the ingredients of 20° C.

FIG. 2 shows the qualitative characteristics of the mixture of the recombinant protein triticain-α obtained by stirring the mixture at a temperature of the individual ingredients of −20°±2° C.

### IMPLEMENTATION OF THE INVENTION

The problem is solved in two stages. Stage 1 is the preparation of a mixture of freeze-dried triticain-α with excipients, which has satisfactory technological characteristics, namely: grindability, flowability, and dosing.

Stage 2 is obtaining a dosage form of triticain-α in the form of solid hypromellose capsules.

At the first stage, the ability of triticain-α in combination with mannitol, polyvinylpyrrolidone MW 12,600 (PVP), and polyvinyl alcohol (PVA), introduced both independently and in a mixture (Table 1) to form lyophilically dried mixtures suitable for subsequent mixing with excipients was studied.

Samples were prepared as follows. To samples concentrated on an Amicon cell with a PM-10 membrane (Millipore) followed by dialysis against phosphate-buffered saline (PBS, pH 7.4, 4° C., 18 h), excipients were added in the ratios shown in Table 1, thoroughly mixed until dissolution and poured into receivers for subsequent lyophilization. Each bottle was corked with an adapter cap for attachment to a freeze dryer. The mixture was then frozen at −70° C. (for at least 2 h) in a Sanyo Ultra Low MDF-U3086S freezer (−70° C.) and placed into a lyophilization unit (Jouan LP-3, France). The lyophilization process was carried out at an evaporator temperature of −50° C. and vacuum of 0.03-0.04 mbar for 24 h.

At the end of the freeze drying process, the bottles were opened, the obtained lyophilizate was removed from them, crushed by impact (using a pestle or dispersant) to avoid grinding, and a powder mixture was obtained. It was found that the only use of mannitol as an excipient led to the formation of brittle, dusty, easily electrified mixtures; while the introduction of only polyvinylpyrrolidone (PVP) or polyvinyl alcohol (PVA) into the mixture did not ensure the fragility of the obtained lyophilisate. The mixtures consisting of triticain-α with polyvinylpyrrolidone or triticain-α with polyvinyl alcohol in the ratios shown in Table 1 were not crushed after lyophilization, separate elastic fibers remained, therefore, the mixtures of triticain-α with these excipients (triticain-α: (PVP), triticain-α: PVA) were not used in further experiments.

TABLE 1

| | Freeze-dried | Ratio of | |
|---|---|---|---|
| | mixture | components | |
| No | composition | (weight parts) | Appearance |
| 1 | 2 | 3 | 4 |
| 1 | Triticain-α | 100 | Lyophilized tablet sub- |
| | | | stance, filamentous |
| 2 | Triticain-α, | 90 | Lyophilized dried mixture |
| | mannitol | 10 | of ingredients as a tablet, |
| | | | filamentous |
| 3 | Triticain-α, | 75 | Lyophilized dried mixture |
| | mannitol | 25 | of ingredients as a tablet, |
| | | | crystalline, with individual |
| | | | inclusions of filaments |
| 4 | Triticain-α, | 50 | Lyophilized dried mixture |
| | mannitol | 50 | of ingredients as a tablet, |
| | | | crystalline |
| 5 | Triticain-α, | 25 | Lyophilized dried mixture |
| | mannitol | 75 | of ingredients as a tablet, |
| | | | crystalline |
| 6 | Triticain-α, | 50 | Lyophilized dried mixture |
| | Mannitol, | 25 | of ingredients as a tablet, |
| | PVP | 25 | crystalline |
| 7 | Triticain-α, | 50 | Lyophilized substance as a |
| | Mannitol, | 30 | tablet, crystalline |
| | PVP | 20 | |
| 8 | Triticain-α, | 50 | Lyophilized dried mixture |
| | Mannitol, | 40 | of ingredients as a tablet, |
| | PVP | 10 | crystalline |
| 9 | Triticain-α, | 50 | Lyophilized dried mixture |
| | Mannitol, | 25 | of ingredients as a tablet, |
| | PVA | 25 | crystalline |
| 10 | Triticain-α, | 50 | Lyophilized dried mixture |
| | Mannitol, | 30 | of ingredients as a tablet, |
| | PVA | 20 | crystalline |
| 11 | Triticain-α, | 50 | Lyophilized substance as a |
| | Mannitol, | 40 | tablet, crystalline |
| | PVA | 10 | |
| 12 | Triticain-α, | 10-90 | Lyophilized dried mixture |
| | PVP | 90-10 | of ingredients as a tablet, |
| | | | filamentous |
| 13 | Triticain-α, | 10-90 | Lyophilized dried mixture |
| | PVA | 90-10 | of ingredients as a tablet, |
| | | | filamentous |

The mixtures with the introduction of PVP into the lyophilisate had the most optimal characteristics. However, it should be noted that the acceptable range of PVP content in the mixture is narrow. E.g., at a PVP content of 75 weight parts in mixtures, significant electrification of the product was noted during grinding, and with PVP contents of 25 weight parts and lower, an increase in the filament content of the freeze-dried product was observed, it did not break down under pressure. Since a brittle product was needed in the future, capable of uniform destruction under minor influences, the PVP content in the selected mixture was within 30-70 weight parts, the optimal ratio was 25 weight parts.

The mixtures of triticain-α: mannitol: PVA with the tested component ratios did not provide the necessary product qualities (brittleness and destructibility), and therefore were not used further.

The selection of excipients for subsequent encapsulation was carried out on the basis of the technological parameters of both a lyophilized mixture of triticain-α with mannitol and PVP, and excipients used in the pharmaceutical industry to prepare oral forms.

Flowability was evaluated in accordance with GPM.1.4.1.0010.15 Powders (State Pharmacopoeia of the Russian Federation), in terms of homogeneity, flowability, bulk density, and moisture content.

Next, we conducted a study of the quality of our freeze-dried powder of the mixture of triticain-α with mannitol, PVP, namely: uniformity, flowability, bulk density (free and tapped), angle of repose; as well as the quantitative protein content, in order to determine the acceptability of the use of the technological stage and to select the ingredients, the parameters of the technological scheme of obtaining a mixture for dosing into capsules.

Such characteristics were studied and calculated as:

Bulk mass (density) is the mass of a unit volume of the powdered or granular material.

Free bulk density. It is determined by freely filling the powder into a container of a certain volume (for example, a measuring cylinder of the ERWEKA model SVM 1), followed by weighing with an accuracy of 0.01 g.

Tapped density. It is determined by measuring the volume of a compacted mass of the powder on an ERWEKA device, model SVM 1.

Carr compressibility index is determined by the difference in the tapped and free densities referred to the tapped density.

The quantitative analysis of the protein (triticain-α) was carried out by HPLC; its content was 49.54±0.25%.

The flowability and angle of repose of the studied powders were determined in accordance with GPM.1.4.2.0016.15 (State Pharmacopoeia of the Russian Federation).

The results obtained on the technological characteristics of the mixture of triticain-α, mannitol, PVP and the excipients used for encapsulation are presented in Table 2.

TABLE 2

| | Free bulk density, | Tapped density, | Flowability, | Angle of repose, |
|---|---|---|---|---|
| Filler | kg/m³ | kg/m³ | g/s | deg |
| 1 | 2 | 3 | 4 | 5 |
| Triticain-α, | 435.80 ± 0.10 | 531.04 ± 0.02 | 10.09 ± 0.02 | 36.6 ± 0.03 |
| Mannitol, PVP | | | | |
| Sodium croscarmellose | 401.5 ± 0.15 | 428.03 ± 0.09 | 7.34 ± 0.02 | 37.9 ± 0.05 |
| Kleptose ® | 392.60 ± 0.30 | 429 ± 0.08 | 6.03 ± 0.02 | 36.9 ± 0.05 |
| Corn starch | 686.0 ± 2.03 | 762.4 ± 1.97 | 1.2 ± 0.02 | 42.7 ± 0.02 |
| Licatab ®C | 583.4 ± 3.05 | 662.3 ± 3.52 | 14.7 ± 0.02 | 32.5 ± 0.03 |
| Prosolv ®SMCC90 | 394.80 ± 0.10 | 451.2 ± 0.02 | 6.06 ± 0.02 | 32.6 ± 0.03 |
| MCC 102 | 603.4 ± 3.05 | 692.1 ± 3.52 | 14.3 ± 0.02 | 30.3 ± 0.03 |

As follows from the data in Table 2, Licatab®C, Prosolv®SMCC90, and MCC-102 were the closest to a mixture of triticain-α, mannitol and PVP in terms of compressibility, and, accordingly, it was concluded that these substances were most suitable to make a powdery mixture suitable for encapsulation. For this reason, sodium croscarmellose and corn starch were excluded from further experiments. Since, according to the literature, Kleptose® is a substance that provides inclusion compounds, a number of experiments were conducted to assess the possibility of further work with it.

When introducing Kleptose® to a mixture of triticain-α with PVP and mannitol in ratios of 10:0.5, 10:1, and 10:2, recommended for creating inclusion compounds [Blouses E. Native and modified cyclodextrins KLEPTOSE multifunctional excipients for molecular encapsulation. Pharmaceutical industry. 2011; 6 (29): 46-48], it was visually noted that the resulting mixture had no satisfactory flowability, and therefore was not further used in research. Prosolv®SMCC90 was also not used in further studies, since it was not possible to obtain visually satisfactory mixtures when introducing it into a mixture of triticain-α, mannitol and PVP in various ratios of ingredients.

Then, powder mixtures were studied, and MCC-102 and Licatab®C were used as auxiliary substances (fillers and dispersants).

As a result of our studies, the optimal ratio of components of the mixture of triticain-α, mannitol, PVP, dispersant and filler was selected.

It was found that a satisfactory quality of the mixture obtained was observed in the following range of mechanical mixtures of triticain-α, mannitol, PVP with the excipients used for encapsulation:

A mixture of triticain-α with PVP and mannitol: 1-10 parts (wt);

microcrystalline cellulose MCC 102: 1-50 parts (wt);

Licatab®C: 98-40 parts (wt).

After analyzing the data obtained, shown in Table 1 and Table 2, suitable excipients were selected and the amount of substances per hydroxypropyl methylcellulose capsule was calculated (Table 3).

TABLE 3

| Composition | Weight (mg) |
|---|---|
| Triticain-α | 20 |
| Mannitol | 10 |
| Polyvinylpyrrolidone MW 12,600 | 10 |
| MCC 102 | 122 |
| Licatab ®C (maltodextrin) | 61 |
| Magnesium stearate | 2 |
| Capsule weight | 85 |
| Total mass of the drug | 310.00 |

The ingredients were mixed in a “Glat” mixer under the following conditions: the mixer rotation speed of 50-250 rpm, the chopper speed of 500-1,500 rpm. With the specified range of operation of the mixing elements of the mixer, a homogeneous mass was obtained. Mixing could be done on any other mixer used to mix solids.

To obtain a homogeneous mixture, a homogeneous mixture of MCC 102 and Licatab-C was first obtained by stirring for 25 min. MCC 102 was first charged as a more crystalline substance and mixed for 5-15 min, then Licatab-C was added and mixed for 20-45 min, achieving uniform distribution. Mixing time and distribution quality were preliminarily evaluated in a model experiment using Licatab painted with red chalk spray paint (Dupli-Color). The assessment was carried out visually.

Then, a mixture of triticain-α with mannitol and PVP, which was not subjected to preliminary grinding, and magnesium stearate (a glidant) was introduced into the mixture under stirring with increased rotation speeds: that of the chopper up to 1,000-5,000 rpm, and that of the mixer up to 100-1,500 rpm. Grinding the mixture and its distribution was carried out in the mixer for 5-15 min; the rotation speed of the chopper was then reduced to the initial one. Stirring was continued for 10-30 min. The temperature of the mixture over the specified time remained constant: −20°±1° C.

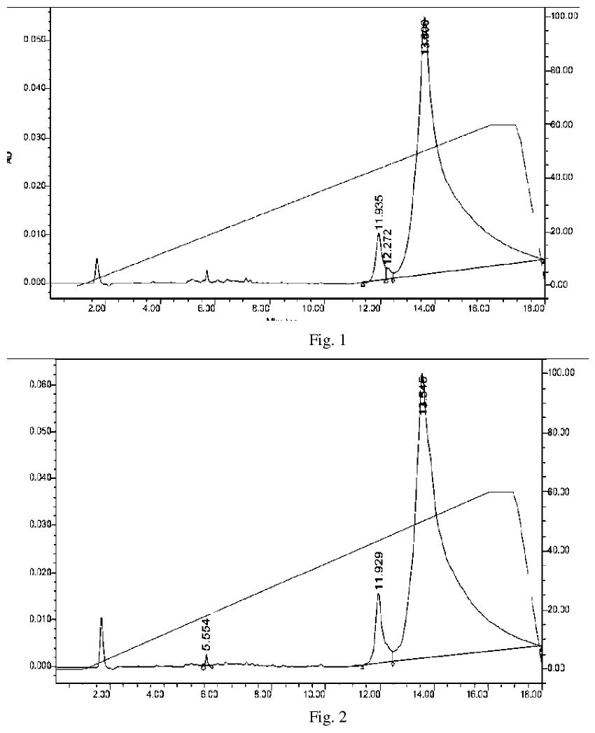

Taking into account the nature of the excipient (a protein), the absence of laboratory mixers with a cooled surface, an experiment was conducted in parallel using mixtures of MCC 102 and Licatab-C cooled down to −20°±2° C. Possible decomposition products were analyzed by HPLC and the quantitative protein content in the mixture obtained with a particular technology was evaluated (FIGS. 1 and 2).

The homogeneity of the protein distribution in the mixture was determined by UV spectrophotometry with a solution of bicinchoninic acid (BCA), after sampling the powder from 5 different places, their subsequent dissolution and centrifugation at 12,600 g. The protein content was within 4.54±0.25% at all points in the mixture.

As follows from the data shown in FIGS. 1 and 2, no traces of decomposition of triticain-α were detected with various methods of obtaining the powder mixture for encapsulation.

After obtaining a homogeneous mixture, the powder was dosed into Vcaps Plus 0 hard capsules using an “ACG-Pam MF 30” tabletop capsule machine.

In addition, we assessed the quality of the obtained product in accordance with the requirements of the GPM.1.4.1.0010.15 Powders (State Pharmacopoeia of the Russian Federation) in terms of homogeneity, flowability, bulk density, and moisture content.

Quality indicators of the mixtures obtained are given below.

The flowability of the powder mass was 17.6±0.3 g/s; its free bulk density was 588.6±3.05 kg/m3, the tapped bulk density was 629.1, the angle of repose was 25.8±0.3, Carr index was 6.4, and the moisture content was 2.91%.

Below we give a specific example of the method.

10 g of mannitol and 10 g of polyvinylpyrrolidone MW 12,600 (PVP) were added to 20 g of the recombinant protein triticain-α, thoroughly mixed until the latter dissolved and poured into receivers for subsequent lyophilization. The bottle was corked with an adapter cap for attachment to a freeze dryer. The mixture was then frozen at −70° C. (for at least 2 h) in a freezer (−70° C., Sanyo Ultra Low MDF-U3086S) and placed into a lyophilization unit (Jouan LP-3, France). The lyophilization process was carried out at an evaporator temperature of −50° C. in vacuum of 0.03-0.04 mbar for 24 h. To obtain a homogeneous mixture, a homogeneous mixture of MCC 102 and Licatab®C, taken in the amount of 122 g and 61 g, respectively, was obtained first, stirred for 25 min with a mixer (“Glat”). MCC 102 was loaded first as a more crystalline mixture and mixed for 5-15 min, and then Licatab®C was added and mixed for 20-45 min, achieving uniform distribution. Then, a mixture of triticain-α with mannitol and PVP in an amount of 40 g, which was not subjected to preliminary grinding, and 2 g of magnesium stearate (a glidant) was introduced into the mixture under stirring with increased rotation speeds: that of the chopper up to 1,000-5,000 rpm, and that of the mixer up to 100-1,500 rpm. Grinding the mixture and its distribution was carried out in the mixer for 5-15 min; the rotation speed of the chopper was then reduced down to the initial one. Stirring was continued for 10-30 min. The temperature of the mixture over the specified time remained constant (20°±1° C.). The yield was 225 g.

The resulting mixture was analyzed for flowability, bulk density (free and tapped), the angle of repose was estimated, and the Carr index was calculated.

The listed indicators were: the flowability of 14.6 g/s; the free bulk density of 578.3±3.05 kg/m3, the tapped bulk density of 654.8, the angle of repose of 31.1±0.3, the Carr index of 11.68. The obtained indicators were considered satisfactory for further development of the technology.

A drug is prepared from the resulting mixture in accordance with the invention.

After obtaining a homogeneous mixture, the powder was dosed into Vcaps Plus 0 hard capsules using an ACG-Pam MF 30 table capsule machine. 1,000 capsules of the following composition per capsule were obtained:

TABLE 4

| Composition | Weight (mg) |
|---|---|
| Triticain-α | 20 |
| Mannitol | 10 |
| Polyvinylpyrrolidone MW 12,600 | 10 |
| MCC 102 | 122 |
| Licatab ®C | 61 |
| Magnesium stearate | 2 |

The method according to the invention allows improving the technological properties of triticain-α and creating a drug and its most suitable dosage form for oral administration.

1. A drug for the treatment of celiac disease based on triticain-α, characterized in that it additionally contains mannitol, PVP, microcrystalline cellulose 102, Licatab®C, magnesium stearate in the following component ratio (weight parts):

| | |
|---|---|
| Triticain-α | 9-20 |
| Mannitol | 5-10 |
| Polyvinylpyrrolidone MW 12,600 | 5-10 |
| Licatab ®C | 26-30 |
| Magnesium stearate | 1-5 |
| MCC 102 | to 100 |

2. The drug for the treatment of celiac disease based on triticain-α according to claim 1, characterized in that it is made in the form of capsules of the following composition per capsule (mg):

| | |
|---|---|
| Triticain-α | 20 |
| Mannitol | 10 |
| Polyvinylpyrrolidone MW 12,600 | 10 |
| MCC 102 | 122 |
| Licatab ®C | 61 |
| Magnesium stearate | 2 |

3. A method for producing the drug of claim 1 for the treatment of celiac disease, which consists in adding the excipients mannitol and polyvinylpyrrolidone MW 12,600 (PVP) to the protein triticain-α, thoroughly mixing, freezing at −70° C. for at least 2 h, lyophilizing at an evaporator temperature of −50° C. in vacuum of 0.03-0.04 mbar for 24 h; microcrystalline cellulose (MCC) 102 and Licatab®C are placed into the mixer, stirred for 25 min to a homogeneous mass, to which the lyophilisate is added; magnesium stearate is added to the resulting mixture, mixed in the mixer for 5-15 min with the chopper operating at 1,000-5,000 rpm, then the chopper rotation speed is reduced down to 500 rpm and stirring is continued for 10-30 min.
